(19) United States
(12) Patent Application Publication
(10) Pub. No.: US 2006/0287721 A1
(43) Pub. Date: Dec. 21, 2006

(54) Artificial cornea

(76) Inventors: David Myung, Mountain View, CA (US); Christopher Ta, Saratoga, CA (US); Nabeel Farooqui, Memphis, TN (US); Curtis W. Frank, Cupertino, CA (US); Won-Gun Koh, Kyunggi Yongin (KR); Jungmin Ko, Mountain View, CA (US); Jaan Noolandi, Palo Alto, CA (US); Michael R. Carrasco, Sunnyvale, CA (US)

Correspondence Address: LUMEN INTELLECTUAL PROPERTY SERVICES, INC., 2345 YALE STREET, 2ND FLOOR, PALO ALTO, CA, 94306, US

(21) Appl. No.: 11/409,218

(22) Filed: Apr. 20, 2006

Related U.S. Application Data
(63) Continuation-in-part of application No. 11/243,952, filed on Oct. 4, 2005.
(60) Provisional application No. 60/673,600, filed on Apr. 21, 2005.
(60) Provisional application No. 60/616,262, filed on Oct. 5, 2004.
(60) Provisional application No. 60/673,172, filed on Apr. 20, 2005.

(51) Int. Cl. A61F 2/14

(52) U.S. Cl. 623/5.15; 264/1.7

(57) ABSTRACT

The present invention provides an artificial corneal implant having an optically clear central core and a porous, hydrophilic, biocompatible skirt peripheral to the central core. In one embodiment, the central core is made of an interpenetrating double network hydrogel and the skirt is made of poly(2-hydroxyethyl acrylate) (PHEA). In another embodiment, both the central core and the skirt are made of interpenetrating double network hydrogels. The artificial corneal implant may also have an interdiffusion zone in which the skirt component is interpenetrated with the core component, or vice versa. In a preferred embodiment, biomolecules are linked to the skirt, central core or both. These biomolecules may be any type of biomolecule, but are preferably biomolecules that support epithelial and/or fibroblast cell survival and growth. Preferably, the biomolecules are linked in a spatially selective manner. The present invention also provides a method of making an artificial corneal implant using photolithography.

### CROSS-REFERENCE TO RELATED APPLICATIONS

This application claims priority from U.S. Provisional Patent Application No. 60/673,600, filed Apr. 21, 2005, which is incorporated herein by reference. This application is a continuation-in-part of U.S. patent application Ser. No. 11/243,952, filed Oct. 4, 2005, which claims priority from U.S. Provisional Patent Application No. 60/616,262, filed Oct. 5, 2004, and from U.S. Provisional Patent Application No. 60/673,172, filed Apr. 20, 2005, all of which are incorporated by reference herein.

### FIELD OF THE INVENTION

The present invention relates generally to corneal implants. More particularly, the present invention relates to artificial corneal implants based on an interpenetrating double network hydrogel core and a peripheral hydrogel skirt.

### BACKGROUND

It is estimated that there are 10 million people worldwide who are blind due to corneal diseases (See e.g. Carlsson et al. (2003) in a paper entitled “Bioengineered corneas: how close are we?” and published in “Curr. Opin. Ophthalmol. 14(4):192-197”). Most of these will remain blind due to limitations of human corneal transplantation. The major barriers for treating these patients are corneal tissue availability and resources, particularly for people in developing countries. To have corneas available for transplantation, a system of harvesting and preserving them must be in place. This requires locating potential donors, harvesting the tissue within several hours of death, preserving the tissue, and shipping it to the appropriate facility within one week. Patients who have had refractive surgery may not be used as donors. Therefore, a shortage of corneas may occur in the future, even in developed countries, as the number of patients undergoing refractive surgery increases. Even among patients who are fortunate enough to receive a corneal transplant, a significant number will develop complications that will result in the loss of vision. The most common complications are graft rejection and failure and irregular or severe astigmatism. In successful cases, the improvement in vision may take many months following the surgery due to graft edema and astigmatism.

A biocompatible artificial cornea with tissue integration and epithelialization can replace the need for a human cornea and provide excellent surgical outcomes. Such an artificial cornea can eliminate the risk of corneal graft rejection and failure, as well as astigmatism, and enable rapid visual recovery. An artificial cornea will ensure an unlimited supply for transplantation anywhere in the world, without the resources required of an eye tissue bank, and eliminate the concern for human cornea shortages due to refractive surgery. Moreover, the technology developed for the artificial cornea can also be applied to the treatment of refractive errors, such as nearsightedness. Through a procedure known as epikeratoplasty (or corneal onlay), a thin polymer can be attached to the cornea to change the refractive index. A biocompatible epithelialized onlay placed over the cornea has an advantage over current technology of laser in situ keratomileusis (LASIK), which requires irreversible corneal tissue removal.

It would be desired to develop an artificial cornea that supports a stable epithelialized surface. Multilayered, stratified epithelial cells would serve as a protective barrier against infections and prevent destructive enzymes from gaining access to the device-cornea interface. The critical requirements for epithelial support of the device are a biocompatible surface for epithelial cellular adhesion and good permeability of glucose and nutrients through the device to support the adherent cells. Other important characteristics of an artificial cornea include optical clarity, biocompatibility, good mechanical strength, and the ability to integrate with stromal tissue.

Accordingly, it would be considered an advance in the art to develop an artificial cornea encompassing these desirable requirements or characteristics.

### SUMMARY OF THE INVENTION

The present invention provides an artificial corneal implant having an optically clear central core and a porous, hydrophilic, biocompatible skirt peripheral to the central core. In one embodiment, the central core is made of an interpenetrating double network hydrogel, with a first network interpenetrated with a second network, and the skirt is made of poly(2-hydroxyethyl acrylate) (PHEA). In another embodiment, both the central core and the skirt are made of interpenetrating double network hydrogels. In both embodiments, the first and second networks of the double network hydrogel are preferably based on biocompatible polymers and at least one of the network polymers is based on a hydrophilic polymer. Preferably, the core and skirt are connected by an interdiffusion zone in which the skirt component is interpenetrated with the core component, or vice versa.

In a preferred embodiment, biomolecules are linked to the skirt, central core or both. These biomolecules may be any type of biomolecule, but are preferably biomolecules that support epithelial and/or fibroblast cell survival and growth. Examples of such biomolecules include, but are not limited to, collagen, fibronectin, laminin, amino acids, carbohydrates, lipids and nucleic acids. Preferably, the biomolecules are linked in a spatially selective manner. For example, the bulk and posterior of the implant's central core may remain unmodified by molecules to maintain passivity to protein adsorption and to enable long-term optical clarity.

The present invention also provides a method of making an artificial corneal implant. With this method, a central core is formed by polymerizing a double network hydrogel. Either simultaneously or in a separate step, a hydrogel-based, biocompatible, hydrophilic skirt is formed by polymerizing a hydrogel under a photolithographic mask with UV light. This photolithographic mask defines the pores in the skirt. The core and skirt are connected by an intediffusion zone in which the skirt component is interpenetrated with the core component, or vice versa. Preferably, either the skirt, central core, or both are then modified with biomolecules.

### BRIEF DESCRIPTION OF THE FIGURES

The present invention together with its objectives and advantages will be understood by reading the following description in conjunction with the drawings, in which:

FIG. 1 shows a schematic of an artificial cornea according to the present invention.

FIG. 2 shows a schematic of formation of an interpenetrating double network hydrogel according to the present invention.

FIG. 3 shows examples of peptides that may be used to modify artificial corneas according to the present invention.

FIG. 4 shows a schematic of biomolecule linkage according to the present invention.

FIG. 5 shows a schematic of tissue integration of an artificial cornea according to the present invention.

FIG. 6 shows a schematic of a method of fabricating an artificial cornea according to the present invention.

FIG. 7 shows a schematic (A) and an actual (B, C) photomask useful for fabricating an artificial cornea according to the present invention.

FIG. 8 shows an example of a photomask (A) and the resulting hydrogel (B, C) formed using a photomask according to the present invention.

FIG. 9 shows a photomicrograph of an artificial cornea according to the present invention.

FIG. 10 shows an example of site-specific modification of a hydrogel with collagen according to the present invention.

FIG. 11 shows examples of cellular growth on an artificial cornea according to the present invention.

FIG. 12 shows examples of tissue integration of an implanted artificial cornea according to the present invention.

### DETAILED DESCRIPTION OF THE INVENTION

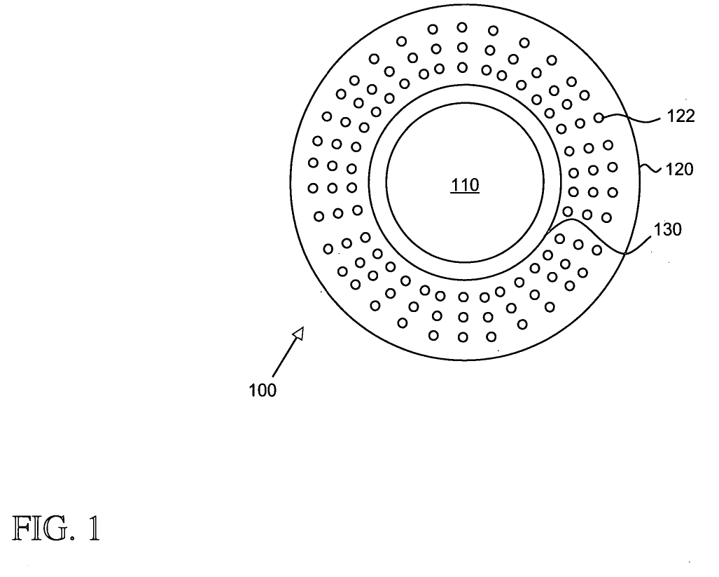

FIG. 1 is a schematic of an artificial corneal implant 100 according to the present invention. Implant 100 contains an optically clear central core 110 and a hydrophilic, biocompatible skirt 120. Skirt 120 contains pores 122 to enable integration with stromal tissue and diffusion of nutrients through implant 100. Optionally, implant 100 may also contain an interdiffusion zone 130, in which central core 110 interpenetrates skirt 120 or vice versa. Exemplary dimensions of implant 100 are as follows: 4.0-12.0 mm total diameter, 3.5-10.0 mm central core diameter, and 15-2000 μm central core and skirt thickness. Pores 122 preferably have a diameter of between about 20 μm and about 200 μm.

Implant 100 preferably has a nutrient diffusion coefficient sufficient to allow passage of nutrients through the artificial cornea. Preferably, central core 110 has a nutrient diffusion coefficient in the range of about 10⁻⁵ cm²/sec to about 10⁻⁷ cm²/sec. Nutrients diffusible through the artificial cornea may be, for example, glucose, growth factors, etc. The diffusion coefficient can be controlled by changing the relative mesh size of the first and second networks.

Central Core

Optically clear central core 110 is preferably made of an interpenetrating double network hydrogel with a first network interpenetrated with a second network. Preferably, the interpenetrating double network is formed by synthesizing a first cross-linked network and then synthesizing a second network in the presence of the first. Since there is no intentional chemical bonding between the two component networks, each network can retain its own properties while the proportion of each network can be varied independently. Such a double network structure is, for example, capable of swelling in water without dissolving and exhibits high mechanical strength as well as high water content, allowing for diffusion of nutrients.

For the purposes of the present invention, the double network hydrogel is based on two biocompatible polymers with at least one of these polymers being hydrophilic. The first network may be based on, for example, poly(ethylene glycol) (PEG), poly(2-hydroxyethyl methacrylate) (PHEMA), collagen, hyaluronic acid, poly(vinyl alcohol) (PVA), poly(vinyl pyrrolidone) (PVP), poly(2-hydroxyethyl acrylate) (PHEA), equivalents thereof, or derivatives thereof. The second network may be based on, for example, poly(acrylic acid) (PAA), poly(acrylamide) (PAAm), poly(hydroxyethyl acrylamide) (PHEAAm), poly(N-isopropylacrylamide) (PNIPAAm), poly(methacrylic acid) (PMAA), poly(2-acrylamido-2-methylpropanesulfonic acid (PAMPS), poly(2-hydroxyethyl methacrylate) (PHEMA), poly(2-hydroxyethyl acrylate) (PHEA), equivalents thereof, or derivatives thereof. Any combination of the described first and second network polymers can be used in a double network structure useful for the present invention.

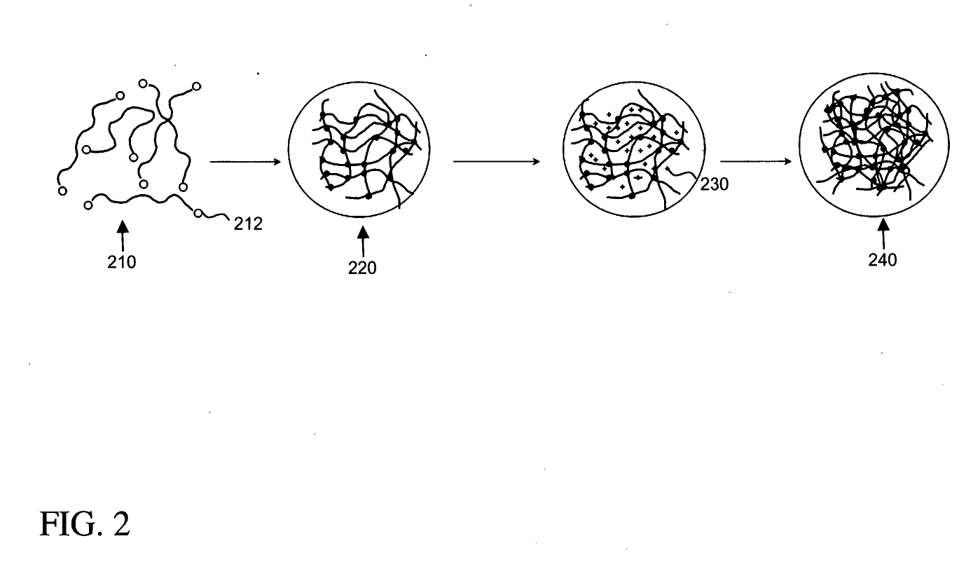

The interpenetrating double-network hydrogel can be synthesized by a (two-step) sequential network formation technique based on UV initiated free radical polymerization (FIG. 2). With this method, a first macromonomer 210 with reactive endgroups 212 is exposed to UV light to form a single network 220. Next, a second monomer 230 is added to the single network 220. Upon addition of second monomer 230, the single network 220 will typically swell. This second monomer is then exposed to UV light to form an interpenetrating double network 240. With this method, the second network composition is typically different from the first. Polymerizing double-network structures by UV light has the advantage that it will enable the use of transparent molds to form artificial corneas of desired shape.

In one embodiment, the polymer polyethylene glycol (PEG) is used as the precursor to the first network. PEG is known to be biocompatible, soluble in aqueous solution, and can be synthesized to give a wide range of molecular weights and chemical structures. The hydroxyl end-groups of the bifunctional glycol can be modified into photo-crosslinkable acrylate end-groups, converting the PEG polymer to PEG-diacrylate (PEG-DA) polymer. Adding a photoinitiator to a solution of PEG-diacrylate in water and exposing to UV light results in the crosslinking of the PEG-diacrylate, giving rise to a PEG-diacrylate hydrogel.

To optimize mechanical and other properties of the double network hydrogel, a variety of acrylic based monomers such as acrylamide, 2-acrylamido-2-methylpropanesulfonic acid, acrylic acid, and methacrylic acid and their derivatives can be used in the synthesis of the second network. In one embodiment, poly(acrylic acid)(PAA) hydrogel is used as the second network. PAA is anionic, containing carboxyl groups that become ionized at pH values above the pKₐ of 4.7. When the carboxyl groups are ionized, their fixed ions repel one another, leading to further swelling. Therefore, hydrogels prepared from PAA exhibit higher equilibrium swelling as pH and AA (acrylic acid) content are increased.

A precursor solution for the first network can be made of purified PEG-DA dissolved in deionized water with 2,2-dimethoxy-2-phenylacetophenone (DMPA) (or 2-hydroxy-2-methyl-propiophenone) as the UV sensitive free radical initiator. The solution can be cast in a mold (e.g. 2 cm in diameter and 250 micrometers in height), covered with glass plates, and reacted under a UV light source at room temperature. Upon exposure, the precursor solution will undergo a free-radical induced gelation and become insoluble in water.

To incorporate the second network, the PEG-based hydrogels may be removed from the mold and immersed in the second monomer solution, such as acrylic acid, containing DMPA (or 2-hydroxy-2-methyl-propiophenone) as the photo-initiator and triethylene glycol dimethacrylate (TEGDMA) as the cross-linking agent for 24 hours at room temperature. The swollen gel may then be exposed to the UV source and the second network will be polymerized inside the first network to form a double-network structure. Other monomers for the second network such as acrylic acid derivatives, methacrylic acid and its derivatives, acrylamide, or 2-acrylamido-2-methylpropanesulfonic acid can be also incorporated into the PEG-based hydrogel using the same or another initiator, crosslinking agent and polymerization procedure.

Instead of PEG, other polymeric materials such as poly(2-hydroxyethyl methacrylate) (PHEMA), poly(vinyl alcohol) (PVA), poly(vinyl pyrrolidone) (PVP), collagen and hyaluronic acid (HA) could be used for the first network. Using these other polymers for the first network, a double-network hydrogel can be synthesized by the same (two-step) sequential network formation technique described above.

For example, to prepare a double network hydrogel using PHEMA as the first network, a PHEMA-based hydrogel could be synthesized by polymerizing a 70/30 (wt/wt) 2-hydroxyethyl methacrylate/distilled water solution containing 0.12 wt % benzoyl peroxide as an initiator. For the gelation, the solution may be reacted in a mold at 60° C. for 24 hours. The second monomer, e.g. acrylic acid, acrylamide, methacrylic acid, or 2-acrylamido-2-methylpropanesulfonic acid is incorporated inside the PHEMA-based hydrogel to form a double network hydrogel by the same process described above.

When PVA is used as the first network, a 10-20% (wt/wt) solution of PVA in water could be prepared at 80 degrees Celsius and cooled to room temperature. Alternatively, a 10-20% (wt/wt) solution of PVA in an 80:20 mixture of dimethyl sulfoxide (DMSO) and water can be heated to 140 degrees Celsius and frozen at −20 degrees Celsius for multiple 24 hour intervals. For PVA crosslinking, a 25% aqueous solution of glutaraldehyde could be combined with 0.01 N sulfuric acid, and a 17% aqueous solution of methanol. This mixture could then be added to the PVA solution and cast in a mold followed by heating at 75 degrees Celsius for 25 minutes. After gelation, the PVA-based hydrogel would be immersed in a solution of a second monomer such as acrylic acid, acrylamide, methacrylic acid, or 2-acrylamido-2-methylpropanesulfonic acid. Using the same polymerization process as described above, the second network can be incorporated inside the PVA-based hydrogel to form a double network structure.

For the synthesis of a double network based on collagen, first, the collagen gel could be formed at physiological conditions by mixing 50% type I, IV or VII collagen, 40% 0.1M NaOH, and 10% 10× concentrated Hank's buffer salt solution (HBSS). Next, 0.02% glutaraldehyde (GTA) may be added in bulk as a cross-linking agent. The final solution can then be cast in a mold before the gel is solidified. The resultant collagen gel may then be immersed in a solution of a second monomer such as acrylic acid, methacrylic acid, derivatives of acrylic acid or methacrylic, acrylamide, or 2-acrylamido-2-methylpropanesulfonic acid. Using the same polymerization process as described above, the second network would then be incorporated inside the collagen gel.

To prepare a double network based on hyaluronic acid (HA), 230 mg of sodium hyaluronan (NaHA) may be mixed with 0.2 M NaOH, pH 13.0, and stirred over ice for 30 minutes. The HA can then be crosslinked with 44 μL of divinyl sulfone in a mold to form a gel. This HA gel may then be immersed in a solution of a second monomer such as acrylic acid, acrylamide, methacrylic acid, or 2-acrylamido-2-methylpropanesulfonic acid. Using the same polymerization process as described above, the second network would be incorporated inside the HA gel.

Attenuated total reflectance/Fourier transform infrared (ATR/FTIR) spectroscopy can be used to monitor the photopolymerization of the hydrogels. The conversion of C═C bonds from the precursor solution to the hydrogel can be monitored by measuring the decrease in terminal C═C bond stretching (RCH═CH₂) at 1635 cm⁻¹ before and after UV exposure. Following synthesis, the double-network hydrogel can be washed extensively in distilled water or PBS to achieve equilibrium swelling and to remove any unreacted components.

The water content of the hydrogels can be evaluated by measuring the weight-swelling ratio. Swollen gels can be removed from the bath, patted dry, and weighed at regular intervals until equilibrium is achieved. The equilibrium water content (WC) can be calculated from the swollen and dry weights of the hydrogel (See e.g. Cruise et al. (1998) in a paper entitled “Characterization of permeability and network structure of interfacially photopolymerized poly(ethylene glycol) diacrylate hydrogels” and published in “Biomaterials 19(14):1287-1294”; and Padmavathi et al. (1996) in a paper entitled “Structural characterization and swelling behavior of poly(ethylene glycol) diacrylate hydrogels” and published in “Macromolecules 29:1976-1979”). All synthesized hydrogels can be stored in sterile aqueous conditions until further use.

Key characteristics of hydrogels such as optical clarity, water content, flexibility, and mechanical strength can be controlled by changing various factors such as the second monomer type, monomer concentration, molecular weight and UV exposure time.

A range of PEG-diacrylate (PEG-DA) double-networks with molecular weights from 575 Da to 14000 Da have been synthesized. It was found that the low molecular weight PEG-DA (<2000 Da) gave rise to gels that were opaque or brittle, whereas the hydrogels made from the higher molecular weight PEG-DA (≧8000 Da) were transparent and flexible. In general and also to prevent phase separation, we found that the molecular weight of PEG should be at least 2000 Da.

In one example, we fixed the concentration of PEG-DA (molecular weight 2000-14000 Da) to 50% (wt/wt) in PBS for the 1^{st} network and changed concentrations of acrylic acid from 15% (v/v) to 60% (v/v). The cross-linking density of the double network hydrogel increased as the molecular weight of PEG decreased and the concentration of acrylic acid increased. We made mechanically strong and transparent hydrogels when the concentration of acrylic acid was in the range of 30% (v/v) to 50% (v/v). In this range of concentration of acrylic acid, the weight ratio of the 1^{st} and 2^{nd} networks ranged from about 1/9 to 3/7. It was also found that incorporation of biomolecules into the double network hydrogel did not change the physical properties of the hydrogel. In one embodiment of the present invention, the double networks have a molar ratio of the first macromonomer ingredient to the second monomer ingredient that is lower than 1/100. In another embodiment of the present invention, the double networks have a molar ratio of the first macromonomer ingredient to the second monomer ingredient in the range of 1/100 to 1/2000.

We have successfully synthesized transparent double-network hydrogels, based on poly(ethylene glycol) (PEG) and acrylic monomers. These double-network hydrogels have better mechanical strength compared to single-network (PEG) hydrogels while maintaining a high water content. For example, crosslinking the second network in the presence of a more densely crosslinked first network leads to a greater than 10-fold increase in tensile strength but less than a 10% decrease in water content.

Skirt

The skirt of the artificial cornea may be made of an interpenetrating double network hydrogel, as described above, or a single network hydrogel. In one embodiment, the skirt is made of PHEA, which is a hydrophilic, biocompatible, and rapidly photopolymerizing network that can be patterned with high fidelity. In addition, PHEA can interpenetrate into another network prior to polymerization to form a “seamless” core-skirt junction. With any skirt material, the central core and skirt of the artificial cornea may be joined together through an interdiffusion zone, in which the central core component interpenetrates the skirt component or vice versa.

Attachment of Biomolecules to the Artificial Cornea

To promote epithelial cell adhesion and proliferation on the nonadhesive central core surface, and to facilitate stromal keratocyte and fibroblast in-growth into the skirt, the surfaces of the core, skirt, or both are preferably modified with biomolecules. Examples of suitable biomolecules include, but are not limited to, cell adhesion-promoting proteins, such as collagen, fibronectin, and laminin, amino-acids (peptides), carbohydrates, lipids, nucleic acids, and the like. Biomolecule modification may be accomplished using two approaches: (1) incorporation of peptides/proteins directly into the polymer during its synthesis and (2) subsequent attachment of peptides/proteins to synthesized hydrogels. The latter approach preferably relies on (a) photoinitiated attachment of azidobenzamido peptides, (b) chemoselective reaction of aminooxy peptides with carbonyl-containing polymers, or (c) photoinitiated functionalization of hydrogels with an N-hydroxysuccinimide group followed by reaction with peptides/proteins.

To incorporate cell adhesion peptides directly into double-network hydrogels, the peptides can be reacted with acryloyl-PEG-NHS to form acrylate-PEG-peptide monomers (See Mann et al. (2001) in a paper entitled “Smooth muscle cell growth in photopolymerized hydrogels with cell adhesive and proteolytically degradable domains: synthetic ECM analogs for tissue engineering” and published in “Biomaterials 22:3045-3051”; Houseman et al. (2001) in a paper entitled “The microenvironment of immobilized Arg-Gly-Asp peptides is an important determinant of cell adhesion” and published in “Biomaterials 22(9):943-955”; and Hem et al. (1998) in a paper entitled “Incorporation of adhesion peptides into nonadhesive hydrogels useful for tissue resurfacing” and published in “J. Biomed. Mater. Res. 39(2):266-276”). These peptide-containing acrylate monomers can be copolymerized with other desired acrylates, including PEG-diacrylates, using standard photopolymerization conditions to form peptide-containing hydrogels. The major advantage of this approach is that the peptide is incorporated directly into the hydrogel, and no subsequent chemistry is needed.

For example, an RGD peptide could be used to form an acrylate-PEG-RGD monomer. This monomer could be copolymerized with PEG-DA in forming the first polymer network or with other acrylates in forming the second polymer network. Peptide incorporation could be confirmed by structural characterization of the hydrogels using attenuated total reflectance/Fourier transform infrared (ATR/FTIR) spectroscopy and X-ray photoelectron spectroscopy (XPS). Additional peptides could be used to make new monomers and corresponding hydrogels.

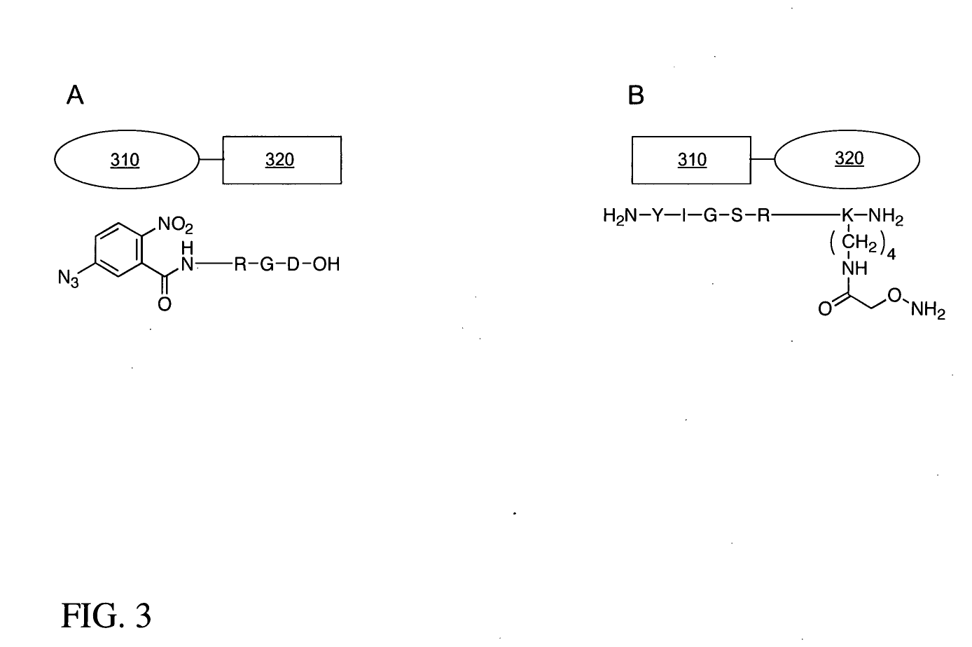

Alternatively, biomolecules may be attached to polymerized hydrogels. In this approach, proteins/peptides are attached with the polymers using (a) photoinitiated reaction of azidobenzamido peptides, (b) chemoselective reaction of aminooxy peptides with carbonyl-containing polymers, or (c) photoinitiated functionalization of hydrogels with an N-hydroxysuccinimide group followed by reaction with peptides/proteins. In each method, the peptides can have two structural features: a recognition sequence that promotes cell adhesion and a coupling sequence/residue. The coupling sequence will feature either an azidobenzoic acid moiety or an aminooxy moiety. FIG. 3 shows two example peptides that were synthesized, an azidobenzamido-RGD peptide (FIG. 3A) and an aminooxy-YIGSR peptide (FIG. 3B), with a generic peptide structure having a coupling sequence 310 and a recognition sequence 320 shown above each example peptide.

The recognition motifs may be the Laminin-derived sequence YIGSR and the fibronectin-derived sequence RGD, each of which has been shown to promote corneal epithelial cell adhesion. The coupling moieties can be attached either directly to the N-termini of the peptides or to the amino group of a C-terminal Lys side chain. The peptides can be synthesized by standard, optimized Boc-chemistry based solid phase peptide synthesis (SPPS). Peptide substrates can be purified by HPLC and identified by electrospray ionization mass spectrometry (ESI-MS).

SPPS gives unparalleled flexibility and control for synthesizing peptides, and it is straightforward to make iterative modifications to independently optimize both the recognition and coupling portions. A major advantage of attachment of peptides after synthesis of the polymers is that it allows combinatorial combination of peptides and polymers to quickly generate large numbers of peptide-decorated hydrogels. For example, five candidate polymers can each be reacted with five peptides to make twenty-five different hydrogels. Moreover, the modular strategy makes it easy to design combinations of different peptides on a single polymer.

Azidobenzamido groups react with light (250-320 μm, 5 min) to generate aromatic nitrenes, which insert into a variety of covalent bonds. Thus, peptides could be modified with 5-azido-2-nitrobenzoic acid or 4-azidobenzoic acid. With this method, candidate polymers are incubated in solutions of the desired peptides and then irradiated with UV light to form covalent linkages between the peptides and the polymers. The advantage of this attachment method is that no special functional groups are necessary on the polymer. The disadvantage is the non-specific nature of the attachment, which may make it difficult to control the amount of peptide on the surface. In addition, possible side reactions include nitrene insertions into other peptides rather than the polymers. Moreover, with certain amino acid residues UV radiation is known to create undesirable structures.

Aminooxy groups react chemoselectively under mild conditions (pH 4-5 buffer, room temperature) to form stable, covalent oxime linkages with ketones. We have made ketone-modified hydrogels by using methyl vinyl ketone (MVK) as one of the co-monomers during the polymerization of the second network. The peptides could be modified with aminooxy acetic acid. Candidate hydrogel polymers can be incubated in mildly acidic solutions of the peptide (0.1 M NaOAc, pH 4.0, 24 h) to effect covalent attachment of the peptide to the polymer. Oxime formation has been used extensively for the chemoselective ligation of biomolecules and proceeds extremely well under mild conditions.

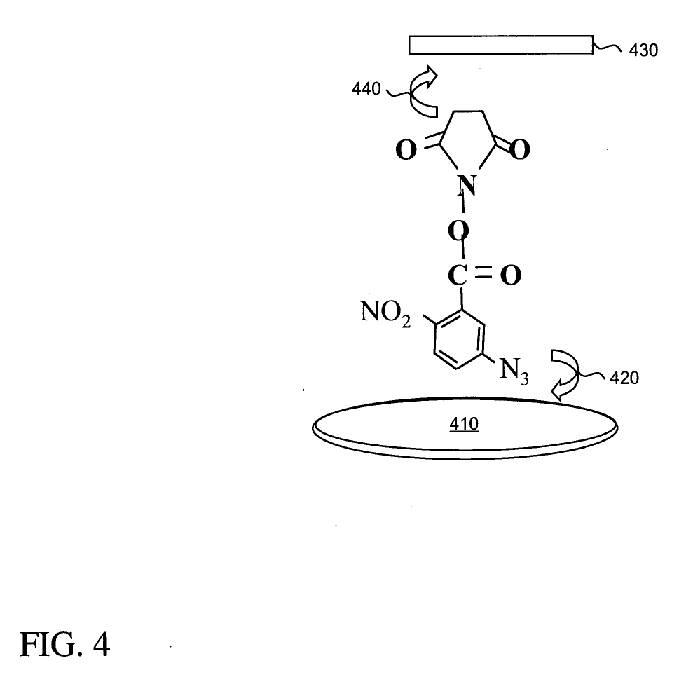

In a preferred embodiment, peptides/proteins are fixed to the artificial cornea photochemically. For the photochemical fixation of peptides/proteins to the hydrogel surfaces, an azide-active-ester chemical containing a photoreactive azide group on one end and an NHS end group (which can conjugate cell adhesion proteins and peptides) on the other end will be used. With this method, shown schematically in FIG. 4, a solution of 5-azido-2-nitrobenzoic acid N-hydroxysuccinimide ester is spread over the hydrogel surface 410. This can be accomplished by dissolving 5 mg of 5-azido-2-nitrobenzoic acid N-hydroxysuccinimide ester in 1 mL of N,N-dimethylformamide (DMF) (See Matsuda et al. (1990) in a paper entitled “Development of micropatterning technology for cultured cells” and published in “ASAIO Transactions 36(3):M559-562”) and spreading the solution over hydrogel surfaces. After air drying the hydrogel, it can then be exposed to UV irradiation 420, for example for 5 minutes. Upon UV irradiation, the phenyl azide group reacts to form covalent bonds with the hydrogel surface 410. The irradiated surfaces will be thoroughly rinsed with solvent to remove any unreacted chemicals from the surface. The hydrogels are then incubated for 24 hours in a solution containing the proteins of interest 430, which react 440 with the exposed NHS end groups.

Preferably, biomolecules attached to the artificial cornea using this method are attached in a site-specific manner, e.g. using photolithography. In a particularly preferred embodiment, the bulk and posterior of the implant's central core will remain unmodified to maintain the intrinsic passivity to protein adsorption of the hydrogel and enable long-term optical clarity. Additionally, pores in the skirt may be selectively tethered with biomolecules that mimic the extracellular matrix of the corneal stroma to encourage tissue integration while minimizing scar formation.

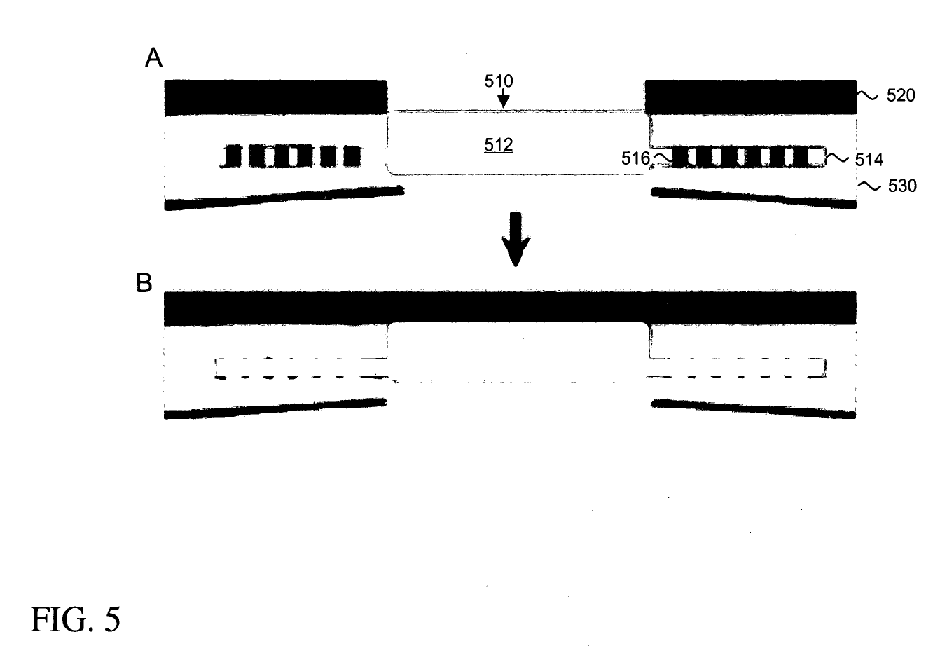

FIG. 5 shows a schematic of how a biomolecularly modified artificial cornea implant would function according to the present invention. Initially, epithelia 520 would be removed and implant 510, with core 512 and skirt 514, with pores 516, will be implanted into stroma 530 (FIG. 5A). In time, epithelial layer 520 will grow over the core 512 and the stroma will grow through the pores 516 to give a fully tissue integrated implant 510 (FIG. 5B). The implant may also have epithelial cells already attached to the implant prior to implantation.

An important aspect of attaching peptides to the surface after polymer synthesis is assessing the success of the attachment. Both analytical and chemical approaches can be used to validate the present methods. Peptide attachment can be confirmed by structural characterization of the hydrogels using ATR/FTIR spectroscopy, XPS and at times amino acid and elemental analysis of the polymers. The attachment strategies can also be validated by using peptides labeled with fluorescent or visible dyes and by use of dynamic contact angle measurements.

Fabrication of an Artificial Cornea

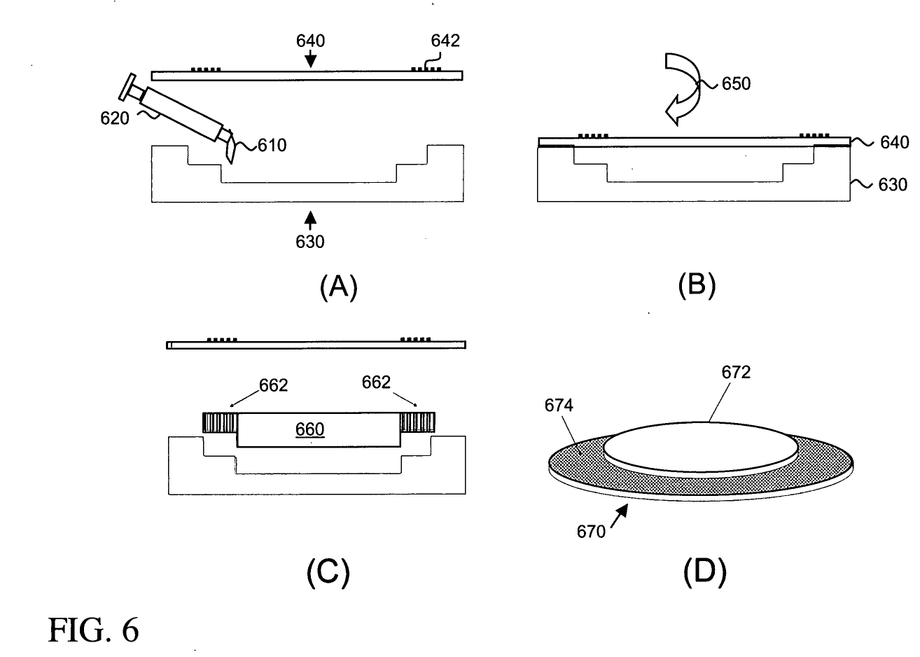

An exemplary protocol for synthesizing an artificial cornea according to the present invention is shown in FIG. 6. Hydrogel precursors 610 are injected with syringe 620 into a two-level Teflon mold 630 and then covered with a photomask 640 with UV blocking discs 642 (FIG. 6A). Either the same or different precursors can be used in the different levels. UV light 650 is then passed through the mask, completely polymerizing the contents of the mold except for the regions in the periphery below UV-blocking discs 642 (FIG. 6B). When removed from the mold, the polymerized hydrogel 660 is left with a pattern of micrometer-sized channels 662 in its periphery (FIG. 6C). A double network hydrogel can then be formed by swelling the entire construct in a second monomer solution, dabbing the excess monomer off, and then exposing the entire swollen hydrogel to UV light. The final result is a construct 670 with a transparent center optic 672 and a porous periphery 674 (FIG. 6C). This construct can then be coated with proteins or peptides by azide-active-ester linkage on its anterior surface as well as in the peripheral skirt region, as described above. The artificial cornea would then be washed thoroughly (e.g. for 1 week in dH₂0) to wash away unreacted monomers before integrating.

In an alternative procedure, the double network core of desired dimensions is synthesized first, washed, and then positioned within a mold under the photomask for the skirt. The skirt monomer (e.g. hydroxyethyl acrylate), photoinitiator and crosslinker, are then injected around the periphery of the core and allowed to interdiffuse into it for a designated period of time (30 seconds to 1 hour). The solution is then exposed to UV light through the photomask to polymerize the skirt around the core; the two are thus connected by the skirt polymer which has diffused into the periphery of the core polymer. A double network skirt can be created by the methods already described, except that after removing excess monomer, only the peripheral region is exposed to UV light to ensure that polymerization is localized to the skirt. (This ensures that a third network is not created in the core region, but a second network is created in the skirt region).

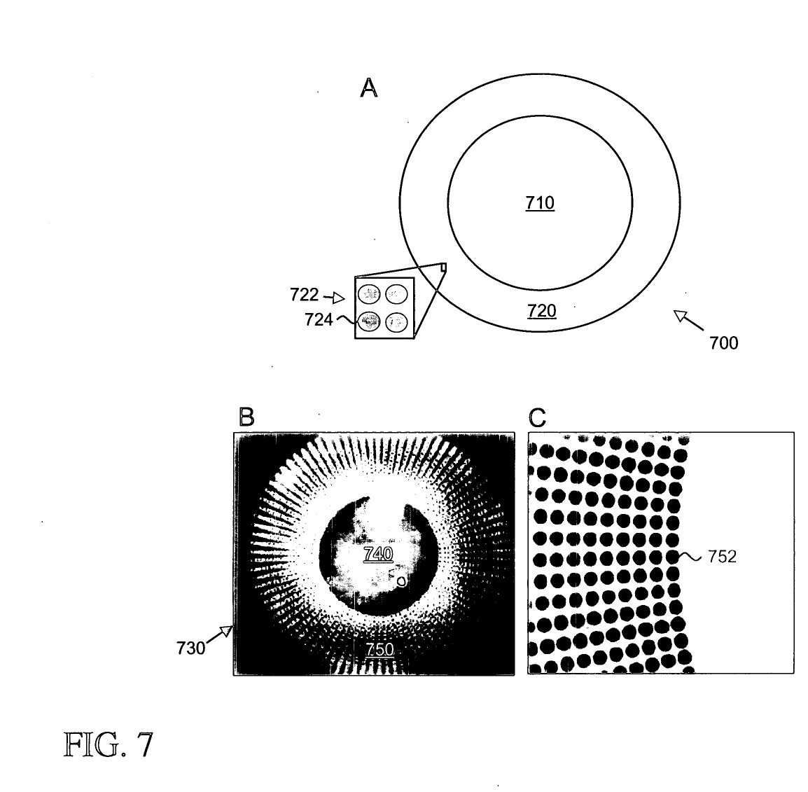

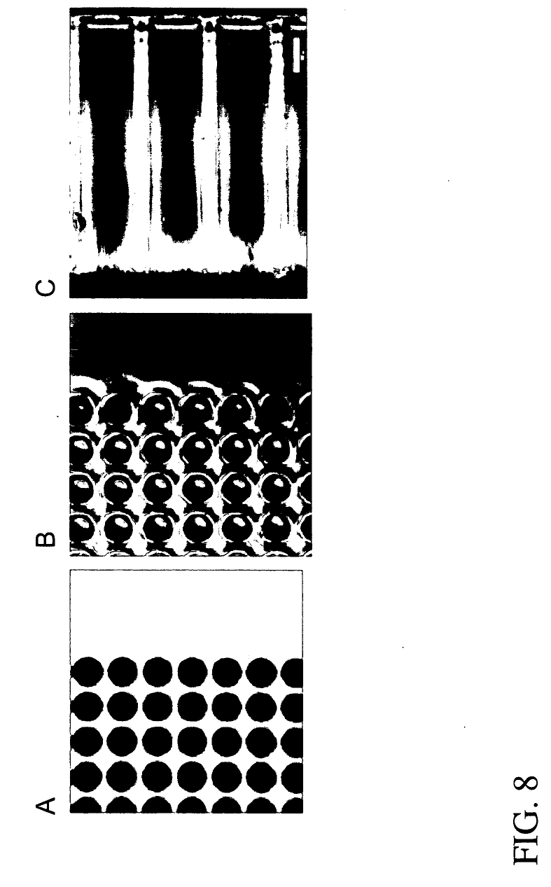

FIG. 7 shows a schematic (A) and an actual (B, C) photolithographic mask that may be used to synthesize porous hydrogel skirts. Mask 700 contains an unmasked central region 710, for forming the central core, and a patterned, masked peripheral region 720, for forming the peripheral skirt. Patterned peripheral region 720 contains UV-blocking disks 724, as shown in insert 722. FIG. 7B shows an actual photolithographic mask 730 that may be used according to the present invention. Discs may be made of any UV-blocking material, including but not limited to chrome, platinum, tungsten, copper, aluminum, gold, or ink. This mask has a 2 cm unpatterned central region 740, and a patterned peripheral region 750 with 60 μm diameter discs 752 spaced 10 μm apart along lines with 1° of separation. Discs 752 can be clearly seen in the magnified view of mask 730, shown in FIG. 7C. While the central region of this mask is 2 cm in diameter, other dimensions are possible. Similarly, other disc dimensions are possible, preferably ranging from about 20 μm to about 200 μm diameter. Any pattern of discs may be used, including but not limited to radial and grid patterns. For example, FIG. 8 shows a photomicrograph of a grid style chrome pattern (A), a representative resulting porous hydrogel after UV irradiation (B) and the porous hydrogel in cross section (C).

### EXAMPLES

Photolithographically Patterned Artificial Cornea

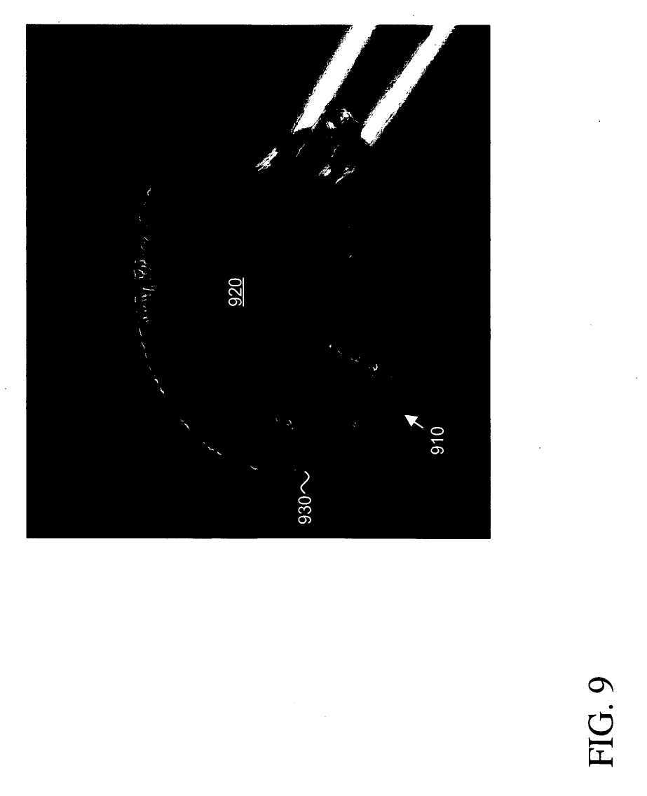

FIG. 9 shows a photomicrograph of a photolithographically patterned artificial cornea 910 with optically clear central core 920 and porous peripheral skirt 930. In this example, the central core was made of a PEG/PAA double network and the skirt was made of PHEA. The PEG/PAA hydrogel was synthesized by a two-step sequential network formation technique based on UV initiated free radical polymerization. A precursor of the first solution was made of purified PEG-diacrylate (MW 8000) dissolved in deionized water with hydroxymethyl propiophenone as the UV sensitive free radical initiator. The solution was cast into a Teflon mold, covered with a glass plate, and reacted under a UV light source at room temperature. Upon exposure, the precursor solution underwent a free-radical induced gelation and became insoluble in water. To incorporate the second network, the PEG hydrogel was removed from the mold and immersed in a 50% v/v acrylic acid solution with 1% v/v hydroxymethyl propiophenone as the initiator, and 1% v/v triethylene glycol dimethacrylate as the cross-linking agent for 24 h at room temperature. The double network hydrogel was then washed extensively in Dulbecco's phosphate buffered saline and allowed to achieve equilibrium swelling. Next, a circular cutting tool was used to cut out a disc, which would become the central core component. The disc was then cast between a glass plate and the center of a photomask. A PHEA precursor solution was then injected around the central optic disc and the monomer was allowed to diffuse into the periphery of the optic for 15 minutes. The photomask was then placed under a UV light source for 60 seconds. The resulting core-skirt construct was then removed from the plates, washed extensively, and stored in phosphate buffered saline until further use.

Site-Specific Biofunctionalization of with Collagen

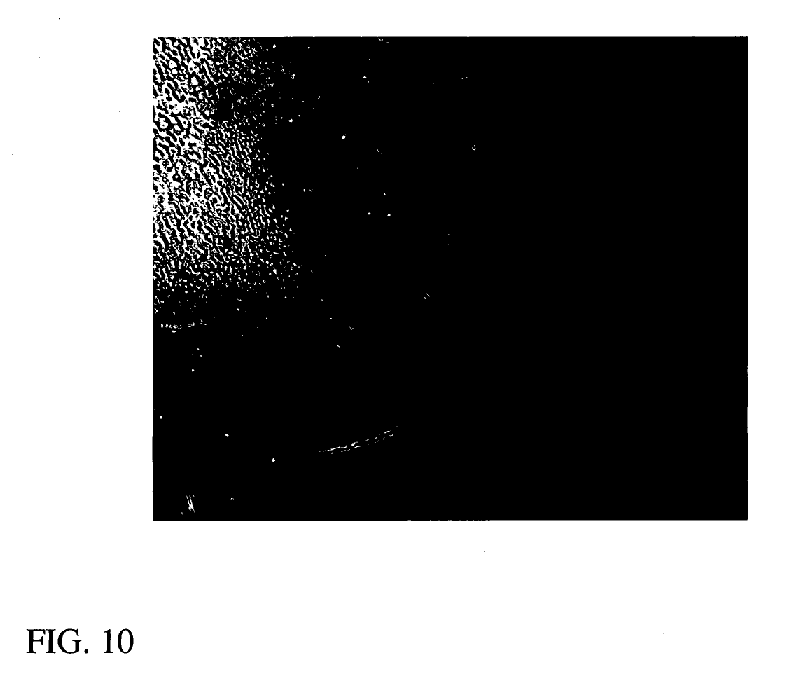

PEG/PAA double network hydrogels were coated with the heterobifunctional photoreactive cross-linker 5-azido-2-nitrobenzoyloxy N-hydroxysuccinimide. The hydrogels were then exposed to a UV light source (75W Xenon Lamp, Oriel Instruments) to induce covalent binding via the azide functional group. This leaves the N-hydroxysuccinimide group exposed for subsequent reaction with the primary amines of collagen type I. Hydrogels functionalized with azide-active-ester and unmodified hydrogels were incubated with 0.1% (w/v) collagen type I (Vitrogen); as a control, PEG/PAA was incubated in deionized water. Fluorescence microscopy was used to visualize the site-specific binding of isothiocyanate (FITC)-labeled collagen to the hydrogels, as shown in FIG. 10. The left side of FIG. 10 shows a gel surface reacted with Collagen-FITC and the right side shows an unreacted gel surface.

Growth of Cells on Hydrogels

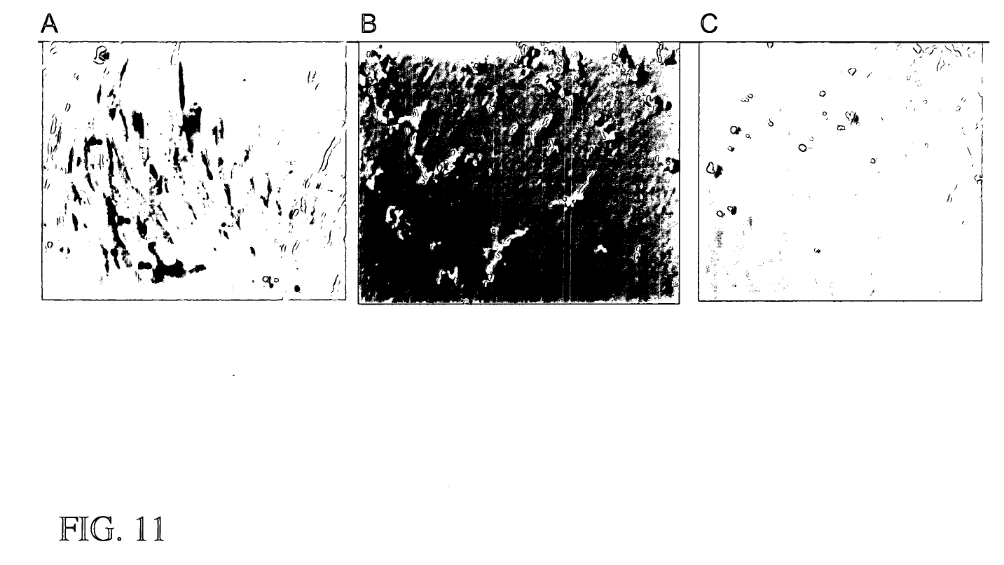

Early passage rabbit corneal epithelial cells screened for epithelial differentiation were seeded on surface-modified PEG/PAA double network hydrogels at a concentration of 1.0×10⁵ cells/cm². The epithelial cells exhibited excellent spreading (>75%) on collagen-bound PEG/PAA networks within 2 hours, achieved confluency within 48 hours, and had migrated over the remainder of the unseeded surface by day 5. A representative photomicrograph of the adherent cells is shown in FIG. 11A. As expected, the unmodified double network did not promote cell attachment or spreading (not shown). In addition, cell spreading was not observed when hydrogels were incubated with collagen type I without prior azide-active-ester functionalization, indicating that little or no physical adsorption of proteins to PEG/PAA had taken place (FIG. 11B).

Early passage corneal fibroblast cells were seeded on collagen type I-modified microperforated PHEA substrates at a concentration of 1.0×10⁵ cells/cm². Cells grew to confluence within 24 hours, as shown in FIG. 11C.

Implantation of Artificial Corneas

We have implanted collagen type I-modified PEG/PAA optics intrastromally for periods of up to 2 months. New Zealand Red rabbits housed in the Animal Research Facility at Stanford University and weighing between 3.5 and 5.5 kg were anesthetized and prepared for surgery using a standard procedure. Before surgery, each rabbit was given an intramuscular injection of ketamine hydrochloride (40 mg/kg), xylazine hydrochloride (4 mg/kg) and glycopyrollate (0.02 mg/kg) with duration of action of 45 min. After this time period, half doses of ketamine hydrochloride (20 mg/kg) and xylazine hydrochloride (2 mg/kg) were administered at 30 min intervals as needed. Once the rabbits were placed under general anesthesia, proparacaine drops were applied to the corneas topically for additional local anesthesia. The sedated animals were then placed in the lateral decubitus position to facilitate surgery on the left eye. The lid margins and the surrounding periorbital area were cleaned with 10% iodine diluted 50:50 with balanced saline solution. Sterile surgical drapes were placed over the upper and lower eyelids of the left eye. Throughout the procedure, corneal drying was prevented by intermittent hydration with balanced saline solution. To facilitate proper suction prior to passage of the microkeratome, the rabbit's eye was slightly proptosed. The handle tip of a sterile, disposable scalpel was inserted into the temporal aspect of the lower conjunctival formix. Using a delicate scooping motion with manual counter-pressure at the 12 o'clock position, the entire globe was proptosed slightly out of the orbit. Proptosis was then maintained by tying a 0-silk suture posteriorly to the equator of the globe. Placement of the hydrogel underneath the epithelial cell layer was achieved by creation of a LASIK flap using a Bausch & Lomb Hansatome microkeratome. Briefly, the 8.5 mm suction ring of the Hansatome apparatus was positioned to achieve adequate vacuum pressure, and then a 160-micrometer stromal flap was created using the microkeratome. The flap was lifted using a LASIK flap spatula, and a sterilized, 3.5 mm diameter hydrogel disc (100 μm thick) was placed onto the stromal bed. The flap was replaced and then sutured to the underlying stroma. Finally, a tarsorraphy (sutured lidclosure) was performed to reduce the chance of implant extrusion. Neomycin, Polymyxin B, and Dexamethasone combination drops were administered three times daily for 10 days post-operatively. Sutures for the cornea flap and eyelids were removed after 7 days.

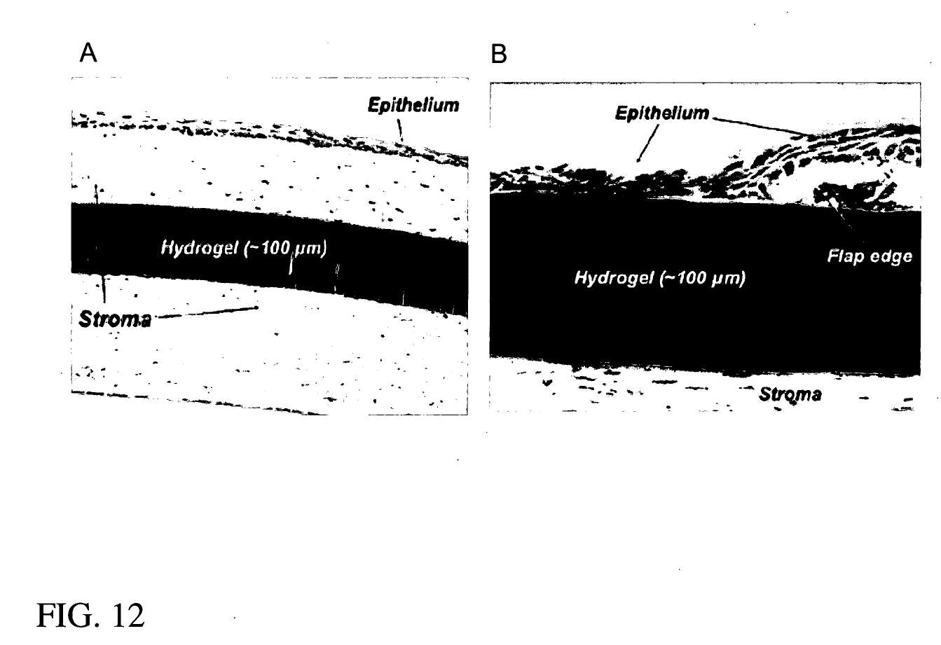

In preliminary studies, the implants were nearly indistinguishable from the surrounding stroma. During a two-week study, collagen type I surface modified PEG/PAA optics (˜100 μm thick, 3.5 mm diameter) were implanted into 8 rabbits to assess the biocompatibility and nutrient permeability of the complete central optic prototype material. The implants were well-tolerated, with no signs of inflammation, epithelial ulceration, or opacification. In one of eight rabbits, the implant extruded due to mechanical factors associated with improper positioning of the optic. Clinical and histological evidence of epithelial and stromal health in these short-term studies demonstrates that the PEG/PAA optics are biocompatible and can facilitate adequate nutrient transport to an overlying epithelium. FIG. 12A shows a histological section demonstrating healthy epithelial growth anterior to a PEG/PAA hydrogel in a rabbit cornea after 14 days.

We have also studied the central optic's capacity to support surface epithelialization in live rabbit corneas. In our study, we implanted 3.5 mm diameter collagen type I-modified PEG/PAA optics into rabbit corneas using the following surgical techniques. A modified corneal onlay procedure was used to implant the PEG/PAA optics. Animals were anesthetized, draped in a sterile fashion, and prepped as described above. Similarly, a LASIK flap was created in the left eye using the Hansotome microkeratome. Once the flap was created, a central hole in the flap was created by the following technique. The flap was lifted using a LASIK flap spatula. A flat metal spatula was then placed under the lifted flap to act as a foundation upon which a 1.5 mm diameter hole was created using a sterile skin biopsy punch. The attached edges were cut using vannas scissors. A 3.5 mm hydrogel button was placed over the stromal bed. The flap was then replaced such that the 1.5 mm flap hole laid over the center of the hydrogel button and was sutured down as described above. The 1 mm rim of stromal tissue was able to secure the implant within the cornea, while the central hole provided an area on the polymer onto which the surrounding epithelium could adhere and migrate. The migration and proliferation of epithelial cells across the polymer surface was evaluated using fluorescein dye to reveal non-epithelialized regions. Wound closure was determined by the lack of fluorescein staining at the end of postoperative week 2 (not shown). FIG. 12B shows histological evidence of multilayered cellular overgrowth on the optic after 14 days in vivo.

As one of ordinary skill in the art will appreciate, various changes, substitutions, and alterations could be made or otherwise implemented without departing from the principles of the present invention. Accordingly, the scope of the invention should be determined by the following claims and their legal equivalents.

What is claimed is:

1. An artificial cornea, comprising:
a) an optically clear central core, wherein said central core comprises an interpenetrating double network hydrogel with a first network interpenetrated with a second network, wherein said first network and said second network are based on biocompatible polymers and wherein at least one of said network polymers is based on a hydrophilic polymer; and
b) a skirt peripheral to said central core, wherein said skirt is hydrophilic, porous, hydrogel-based, and biocompatible.

2. The artificial cornea as set forth in claim 1, wherein said skirt comprises a double network hydrogel with a first network interpenetrated with a second network, wherein said first network and said second network are based on biocompatible polymers and wherein at least one of said network polymers is based on a hydrophilic polymer.

3. The artificial cornea as set forth in claim 1, wherein said skirt comprises poly(2-hydroxyethyl acrylate) (PHEA).

4. The artificial cornea as set forth in claim 1, wherein said skirt, said central core, or said skirt and said central core further comprise biomolecules covalently linked to said skirt, said central core, or said skirt and said central core.

5. The artificial cornea as set forth in claim 4, wherein said biomolecules are selected from the group consisting of collagen, fibronectin, laminin, amino acids, carbohydrates, lipids, and nucleic acids.

6. The artificial cornea as set forth in claim 4, wherein said covalent linkage comprises an azide-active-ester linkage.

7. The artificial cornea as set forth in claim 4, wherein said covalent linkage comprises a 5-azido-2-nitrobenzoic acid N-hydroxysuccinimide ester linkage or a derivative thereof.

8. The artificial cornea as set forth in claim 4, wherein said biomolecules are linked in a spatially selective manner.

9. The artificial cornea as set forth in claim 4, further comprising epithelial cells adhered to said biomolecules.

10. The artificial cornea as set forth in claim 1, wherein said artificial cornea has a nutrient diffusion coefficient sufficient to allow passage of nutrients through said artificial cornea.

11. The artificial cornea as set forth in claim 1, wherein said central core has a nutrient diffusion coefficient in the range of about 10⁻⁵ cm²/sec to about 10⁻⁷ cm²/sec.

12. The artificial cornea as set forth in claim 1, wherein said pores have a diameter of between about 20 μm and about 200 μm.

13. The artificial cornea as set forth in claim 1, wherein said first network is based on poly(ethylene glycol) (PEG), poly(2-hydroxyethyl methacrylate) (PHEMA), collagen, hyaluronic acid, poly(vinyl alcohol) (PVA), poly(vinyl pyrrolidone) (PVP), poly(2-hydroxyethyl acrylate) (PHEA), or derivatives thereof.

14. The artificial cornea as set forth in claim 1, wherein said second network is based on poly(acrylic acid) (PAA), poly(acrylamide) (PAAm), poly(hydroxyethyl acrylamide) (PHEAAm), poly(N-isopropylacrylamide) (PNIPAAm), poly(methacrylic acid) (PMAA), poly(2-acrylamido-2-methylpropanesulfonic acid) (PAMPS), poly(2-hydroxyethyl methacrylate) (PHEMA), poly(2-hydroxyethyl acrylate) (PHEA) or derivatives thereof.

15. The artificial cornea as set forth in claim 1, wherein said first network is polymerized from macromonomers and said second network is polymerized from monomers, wherein the molar ratio of the first network macromonomer to the second network monomer is lower than 1/100.

16. The artificial cornea as set forth in claim 1, wherein said first network is polymerized from macromonomers and said second network is polymerized from monomers, wherein the molar ratio of the first network macromonomer to the second network monomer is in the range of 1/100 to 1/2000.

17. The artificial cornea as set forth in claim 1, wherein the weight ratio between said first network and said second network is in the range of 1/9 to 3/7.

18. The artificial cornea as set forth in claim 1, wherein said first network is based on poly(ethylene glycol)-diacrylate (PEG-DA) and said second network is based on poly(acrylic acid) (PAA).

19. The artificial cornea as set forth in claim 18, wherein the concentration of poly(acrylic acid) (PAA) is in the range of 30% (v/v) to 50% (v/v).

20. The artificial cornea as set forth in claim 1, wherein said double network hydrogel is based on polyethylene glycol (PEG) with a molecular weight of 2000 Da or higher.

21. The artificial cornea as set forth in claim 1, wherein said double network hydrogel is based on polyethylene glycol (PEG) with a molecular weight of 2000 Da to 14000 Da.

22. The artificial cornea as set forth in claim 1, wherein said core and said skirt are connected by an interdiffusion zone.

23. The artificial cornea as set forth in claim 22, wherein material forming said core interpenetrates material forming said skirt, or wherein material forming said skirt interpenetrates with material forming said core.

24. A method of fabricating the artificial cornea as set forth in claim 1, comprising:
a) forming said central core by polymerizing said double network hydrogel;
b) forming said porous, hydrogel-based, biocompatible, hydrophilic skirt by polymerizing said hydrogel under a photolithographic mask with UV light, wherein said photolithographic mask defines said pores in said skirt.

25. The method as set forth in claim 25, further comprising site-specifically modifying the anterior surface of said central core with a biomolecule.

26. The method as set forth in claim 25, further comprising modifying said skirt with a biomolecule.
